(19) United States
(12) Patent Application Publication
(10) Pub. No.: US 2025/0314580 A1
(43) Pub. Date: Oct. 9, 2025

(54) Finish saturator for enhanced paint finish testing

(71) Applicant: Kellon Darwin Johnson, Granite Falls, WA (US)

(72) Inventor: Kellon Darwin Johnson, Granite Falls, WA (US)

(21) Appl. No.: 18/628,795

(22) Filed: Apr. 7, 2024

(51) Int. Cl. G01N 19/04; G01N 33/32

(52) U.S. Cl. CPC G01N 19/04; G01N 33/32

(57) ABSTRACT

A Finish Saturator for testing weather resistance and adhesion properties of paint finishes, especially for automotive and aerospace industry applications, is disclosed. The device includes a 6″ square, 3-mil thick polyurethane plastic body with a non-residual adhesive perimeter that forms a 5.0″ square non-adhesive center pocket for a water-soaked cloth. The design facilitates standardized testing conditions, enhancing result reliability and reducing testing resources.

### BACKGROUND OF THE INVENTION

In the automotive and aerospace industries, testing the adhesion and weather resistance of paint finishes is crucial to ensuring the longevity and durability of paint jobs under a variety of environmental conditions. The current traditional methods, which often involve manually crafted makeshift bags from materials such as plastic and duct tape, result in inconsistent test conditions and unreliable results. This necessitates a standardized, efficient, and dependable testing method to enhance quality control and product durability.

### FIELD OF THE INVENTION

This invention pertains to the field of materials testing and specifically introduces a device and method dedicated to assessing the weather resistance and adhesion properties of paint finishes applicable in automotive and aerospace industries.

### SUMMARY OF THE INVENTION

The Finish Saturator is devised to overcome the challenges mentioned above by presenting a standardized and user-friendly device aimed at testing the adhesion and weather resistance of paint finishes. The invention embodies a square composed of polyurethane plastic, featuring a pre-applied, water-resistant, non-residual adhesive along its perimeter. This adhesive frame a central pocket designed to secure a water-soaked cloth against the paint finish, facilitating standardized testing, enhancing result consistency and reliability, and reducing required testing time and materials.

### BRIEF DESCRIPTION OF THE SEVERAL VIEWS OF THE DRAWINGS

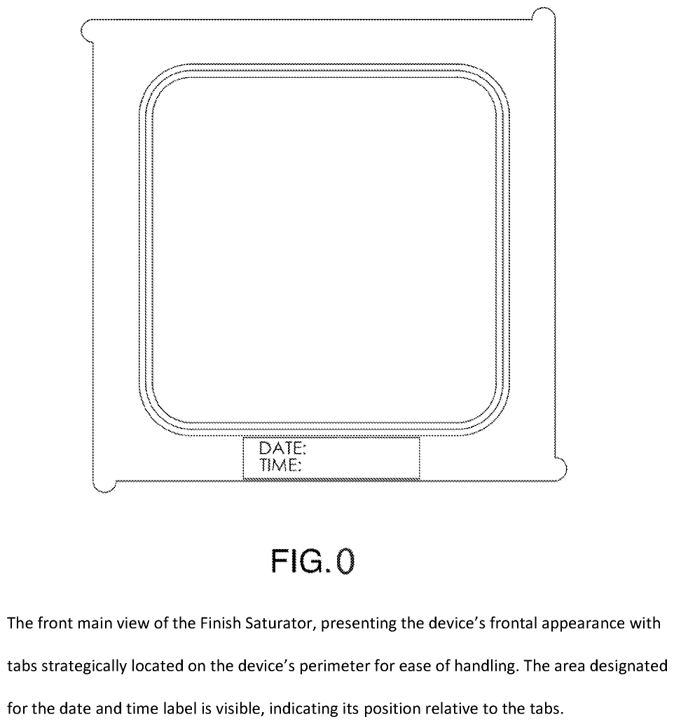

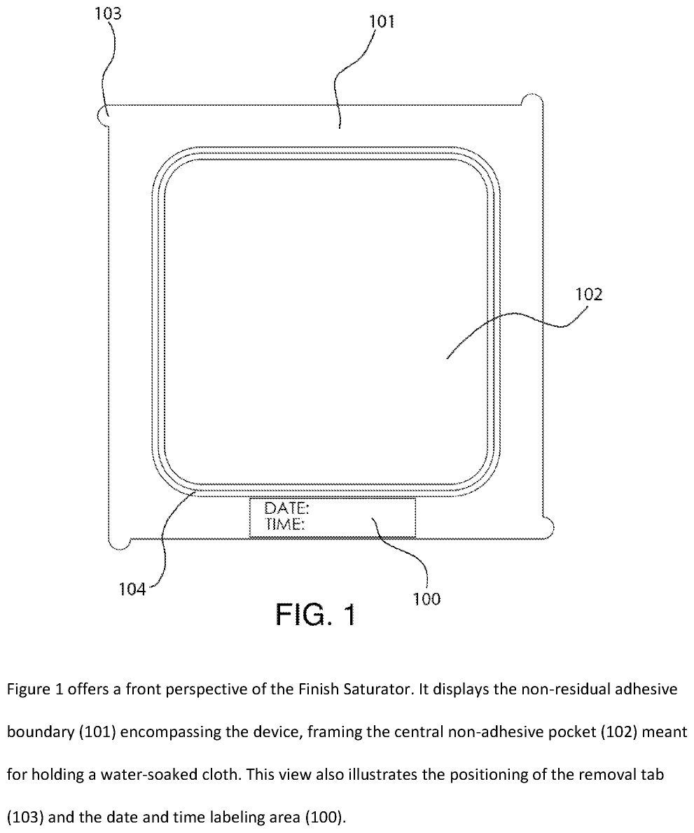

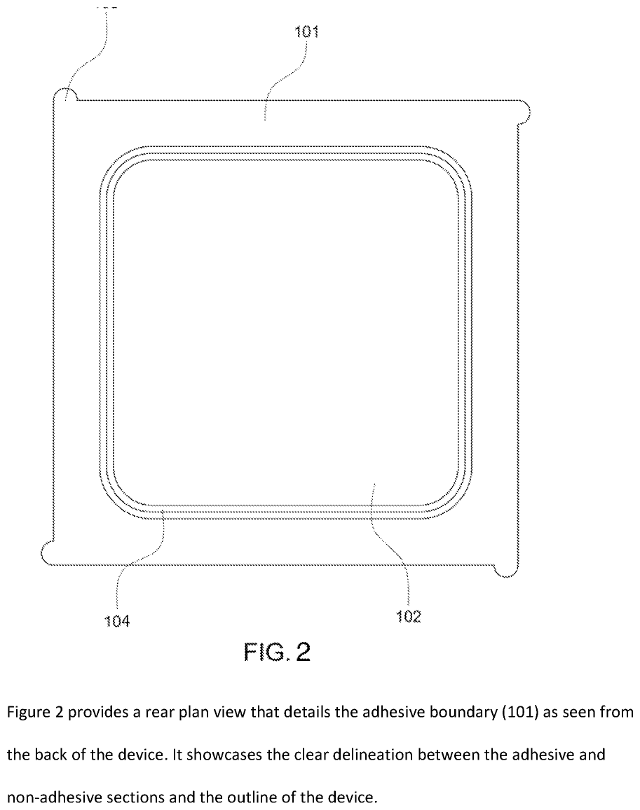

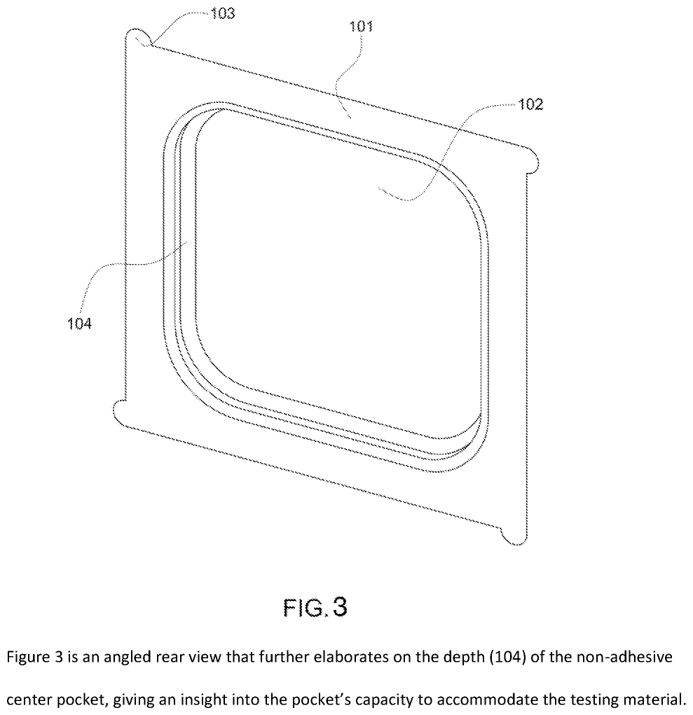

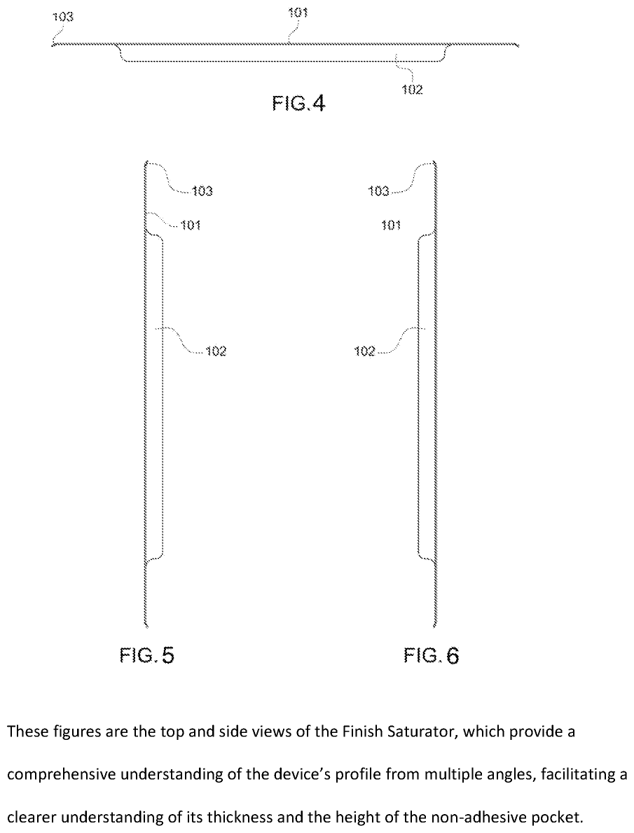

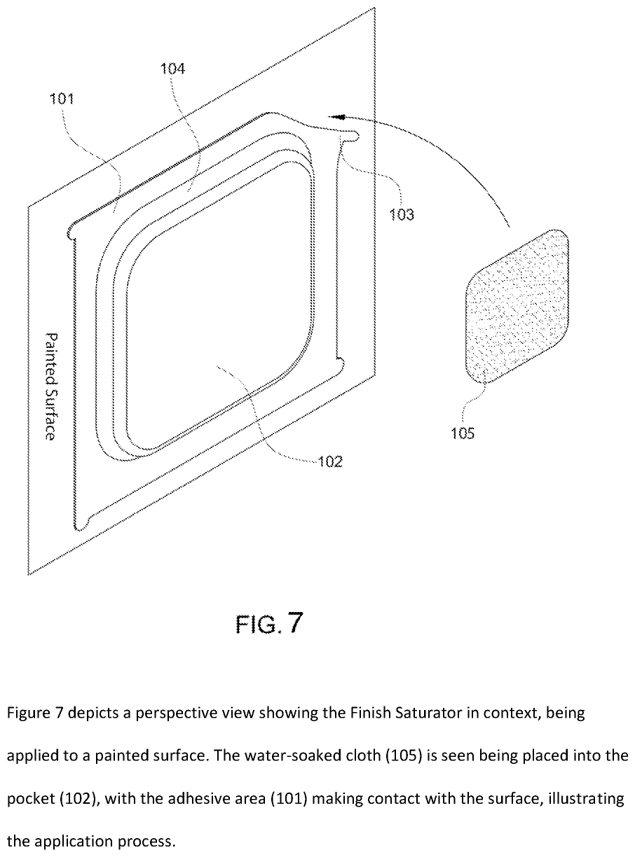

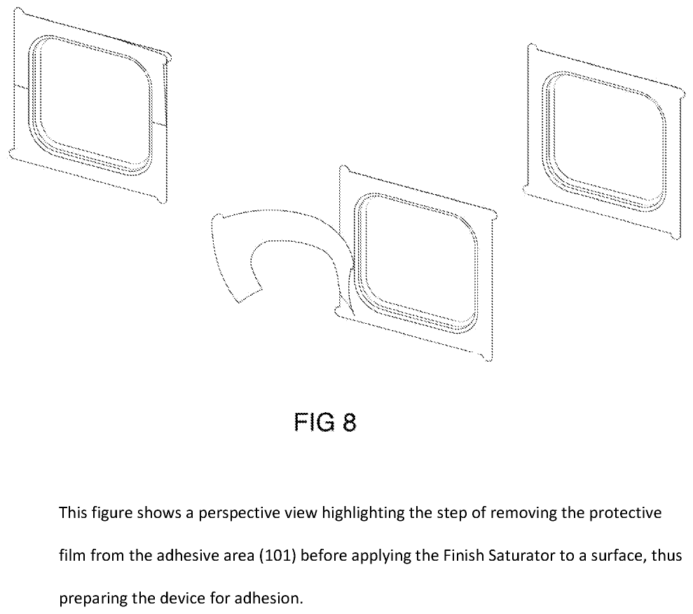

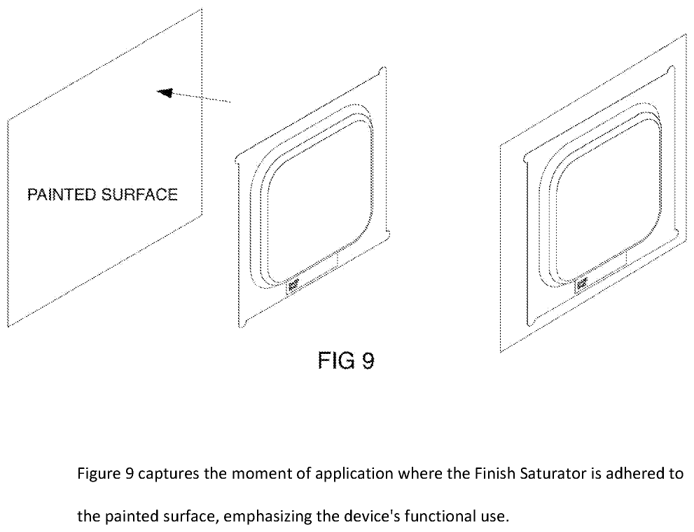

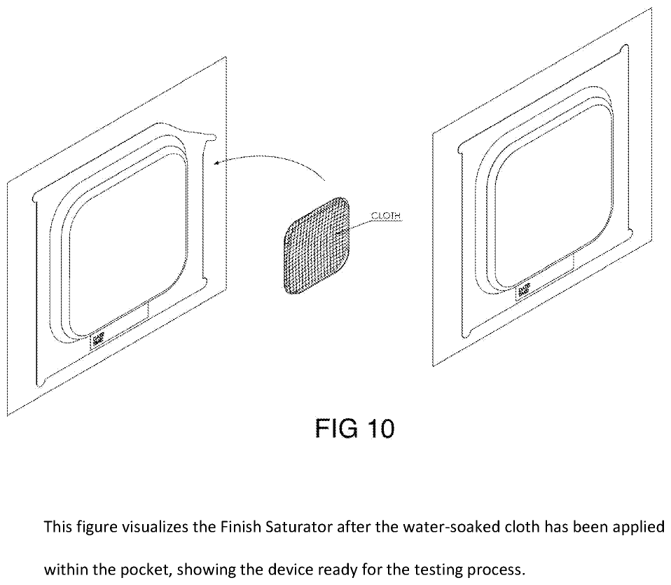

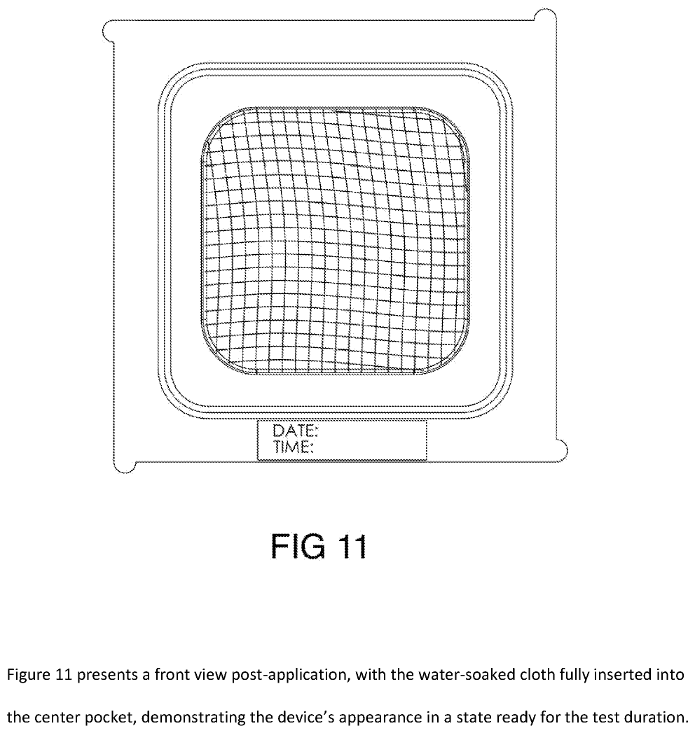

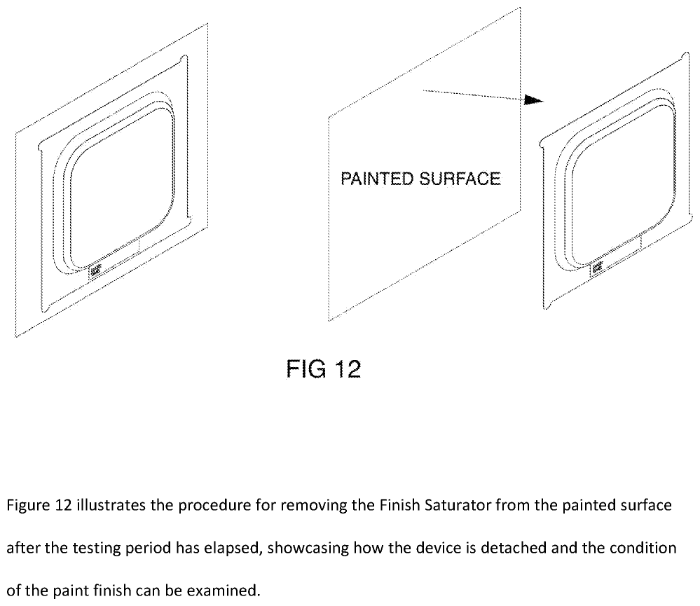

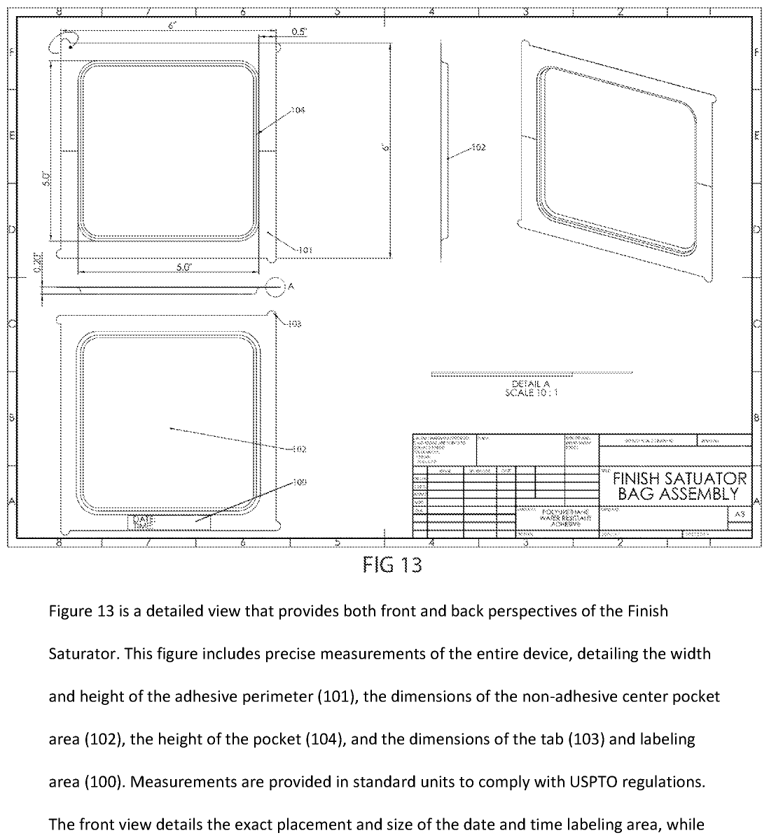

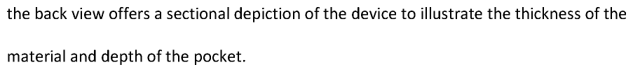

The figures provide multiple views of the Finish Saturator, detailing its structure and application process:
Sheet 1/12, FIG. 0: Front Main View of the Finish Saturator illustrating the placement of tabs and date/time label.
Sheet 2/12, FIG. 1: Front View displaying the adhesive perimeter (101) and center pocket (102).
Sheet 3/12, FIG. 2: Back Plan View showing the non-adhesive center and adhesive boundary.
Sheet 4/12, FIG. 3: Back Angled View indicating the depth of the center pocket (104).
Sheet 5/12, FIGS. 4-6: Top and Side Views presenting the device's two-dimensional aspects.
Sheet 6/12, FIG. 7: Perspective View depicting the application process onto a painted surface with a water-soaked cloth (105).
Sheet 7/12, FIG. 8: Perspective View detailing the removal of the protective film from the adhesive area (101).
Sheet 8/12, FIG. 9: Perspective View showing device adherence to the painted surface.
Sheet 9/12, FIG. 10: Perspective View after cloth application.
Sheet 10/12, FIG. 11: Front View post-application showing cloth insertion.
Sheet 11/12, FIG. 12: Perspective View illustrating the device's removal post-testing.
Sheet 12/13, FIG. 13: detailed view that provides both front and back perspectives of the Finish Saturator.

### DETAILED DESCRIPTION OF THE INVENTION

### Technical Specifications:

Material: Constructed from 3-mil thick polyurethane plastic (102), known for durability and environmental resistance.
Dimensions: The device measures 6″ square with 0.5″ tabs (103) for removal. The center pocket area is 5.0″ square (102), with a 0.5″ adhesive width (101) and a pocket depth of 0.20″ (104).
Adhesive: A perimeter adhesive (101) is water-resistant and leaves no residue.
Labeling Area: A side area measuring 0.50″ by 2.0″ for date and time annotations (100).

### Innovative Features:

Standardized Design: Facilitates consistent testing for adhesion and weather resistance.
Ease of Use: Simplified setup process due to pre-applied adhesive and designated pocket for testing materials.
Consistency: Uniform testing conditions for reliable results.
Time and Material Savings: More efficient testing means for the industry.

Utility and Industrial Applicability: Tailored for industries that value the integrity and endurance of paint finishes, such as automotive and aerospace. The design emphasizes consistency, reliability, and testing efficiency, which promotes quality control and augments research and development, providing an improved alternative to conventional methods.

Conclusion: The Finish Saturator constitutes a pivotal innovation in the technology of paint finish testing. It is a critical tool for quality and durability assurance in paint finishes within the automotive and aerospace sectors, contributing to the progression of quality control practices.

1. A paint finish testing device, comprising: a square body constructed from 3-mil thick polyurethane plastic; a central pocket area demarcated by a water-resistant, non-residual adhesive bordering the square body, leaving a non-adhesive section to constitute the pocket; where the square body is approximately 6 inches per side, and the central pocket area is approximately 5 inches per side; further including tabs on opposing ends of the square body to facilitate application and removal from a test surface.

2. The device of claim 1, wherein the central pocket is designed to accommodate a water-soaked cloth for testing the adhesion properties of a paint finish on the test surface.

3. The device of claim 1, wherein the depth of the central pocket is approximately 0.20 inches, to securely hold the water-soaked cloth in place against the paint finish.

4. The device of claim 1, further comprising a labeling area on the surface of the square body, opposite the adhesive, for recording the date and time of application to the test surface.

5. A method for testing the weather resistance and adhesion properties of a paint finish, the method comprising:
providing a testing device as defined in claim 1.
soaking a cloth in water and inserting the cloth into the central pocket of the device.
applying the device to a painted surface such that the water-soaked cloth is in contact with the paint finish.
leaving the device in place for a duration ranging from 1 to 24 hours.
removing the device and assessing the adhesion properties of the paint finish based on the interaction with the water-soaked cloth.

6. The device of claim 1, wherein the water-resistant, non-residual adhesive is applied to a width of 0.5 inches from the edge towards the center on each side of the square body, ensuring a secure and residue-free attachment to and removal from the test surface.

7. A standardized paint finish testing kit comprising:
one or more testing devices as defined in claim 1.
instructions for applying the testing device to a painted surface.
guidelines for assessing the adhesion properties of the paint finish following the test.

8. The method of claim 5, further including the step of utilizing the labeling area on the testing device to record the date and time of application, thereby facilitating tracking and documentation of the testing process.

9. A paint finish testing device designed for industrial application in the automotive and aerospace industries, wherein the device's dimensions, adhesive properties, and method of use are specifically optimized for testing the weather resistance and adhesion properties of paint finishes in these industries.

10. The device of claim 1, where the polyurethane plastic and adhesive materials are selected for their compatibility with automotive and aerospace paint finishes, ensuring that the testing process does not alter or damage the paint finish beyond the intended testing interaction.
